(19) United States
(12) Patent Application Publication
(10) Pub. No.: US 2022/0235887 A1
(43) Pub. Date: Jul. 28, 2022

(54) SYSTEM FOR CONNECTING TUBING

(71) Applicant: GA HEALTH COMPANY LIMITED, Shek Mun (HK)

(72) Inventor: Kenneth McCabe, Shek Mun (HK)

(73) Assignee: GA HEALTH COMPANY LIMITED, Shek Mun (HK)

(21) Appl. No.: 17/159,890

(22) Filed: Jan. 27, 2021

(51) Int. Cl. F16L 19/02

(52) U.S. Cl. CPC F16L 19/0237; F16L 19/0206

(57) ABSTRACT

A system and method for joining tubing of different sizes including a first connector including a first and a second projection and a lumen extending therethrough. The first projection has an outer diameter larger than an outer diameter of the second projection. A first coupling includes a first and second coupling member. The first coupling member has a first body including a first aperture extending therethrough along a length thereof, the first body including threads. The second coupling member includes a second body having a second aperture extending therethrough along a length thereof. The second body includes threads complimentary with the threads of the first coupling member permitting the first and second member to be threadedly engaged, and the first aperture has a diameter larger than a diameter of the second aperture.

### TECHNICAL FIELD

The disclosure relates to a connector for joining flexible tubing. More particularly a connector for securely joining flexible tubing or different sizes.

### BACKGROUND

A secure connection of flexible tubing is vital in preventing leakage, especially in health care and laboratory environments. Often flexible tubing of different sizes need to be joined together in order to complete a fluid communication circuit. Connectors of the prior art tend to have complex latching arrangements that are difficult to manufacture and use. Prior art solutions also include the use of couplings with barded ends of different sizes. However, if the barb is too tight, then assembly and disassembly of the tubing and connector is difficult. If the barb is too loose, then there is a risk of the tubing separating from the connector. Snap fittings that include latching members are also employed however, they can be difficult to secure and remove. Since such fittings rely on deflecting of plastic material, multiple secure and removal cycles can result in permanent deformation and thus damage and weaken the connection. In addition, repeated and continued use of the deflecting members may be so that the connecting members do not effectively return to their unbiased state. This, in turn, can result in a reduced latching force that can be inadvertently overcome.

It would be desirable, therefore, to provide a system for coupling tubing that is easy to manufacture, assemble and use that provides consistent connection between tubing of differing sizes.

### SUMMARY

The present disclosure provides a system for joining tubing of different sizes including a first connector including a first and a second projection and a lumen extending therethrough. The first projection has an outer diameter larger than an outer diameter of the second projection. A first coupling includes a first and second coupling member. The first coupling member has a first body including a first aperture extending therethrough along a length thereof, the first body including threads. The second coupling member includes a second body having a second aperture extending therethrough along a length thereof. The second body includes threads complimentary with the threads of the first coupling member permitting the first and second member to be threadedly engaged, and the first aperture has a diameter larger than a diameter of the second aperture.

The present disclosure also provides a system for joining tubing of different sizes including a first connector including first and second projection and a lumen extending therethrough. The first projection has an outer diameter larger than an outer diameter of the second projection. A second connector has a third and fourth projection and a lumen extending therethrough. The third projection having an outer diameter substantially similar to the outer diameter of the second projection and the fourth projection has an outer diameter different from the outer diameter of the first and second projections. A first coupling includes a first and second coupling member. The first coupling member includes a first aperture extending therethrough along a length thereof. The first coupling member has a first joining element. The second coupling member includes a second aperture extending therethrough along a length thereof. The second coupling member includes a second joining element complimentary with the first joining element of the first coupling member permitting the first and second coupling members to be secured together. The first aperture has a diameter larger than a diameter of the second aperture. A second coupling has a third and a fourth coupling member, and the third coupling member have an aperture therethrough with a diameter substantially the same as the diameter of the aperture in the second coupling member. The fourth coupling member has an aperture with a diameter different from the diameter of the apertures in the first, second, and third coupling members.

The present disclosure further provides a method for joining tubing of different sizes including, providing a first connector including first and second projection and a lumen extending therethrough, the first projection having an outer diameter larger than an outer diameter of the second projection;

providing a first coupling including a first and second coupling member, the first coupling member including a first aperture extending therethrough along a length thereof, the first coupling member having a threaded portion, the second coupling member including a second aperture extending therethrough along a length thereof, the second coupling member including a threaded portion complimentary with the threads of the first coupling member threaded portion permitting the first and second coupling members to be threadedly engaged, and the first aperture having a diameter larger than a diameter of the second aperture;

inserting a first tube first end into the first projection;

inserting a second tube first onto the second projection, wherein the second tube has an inner diameter size different that an inner diameter of the first tube;

inserting the first tube through the aperture in the first coupling member;

inserting the second tube through the aperture in the second coupling member; and

advancing the first coupling and second coupling member and threading the first coupling and second coupling member together to secure the first tube and second tube to the first connector.

### BRIEF DESCRIPTION OF THE DRAWINGS

FIG. 1 is a perspective view of the system showing three pieces of tubing connected.

FIG. 2 is an exploded perspective view of the system of FIG. 1.

FIG. 3 is a cross-sectional view taken along line 3-3 of FIG. 1.

FIG. 4 is a perspective view of coupling members being placed on the tubing pieces.

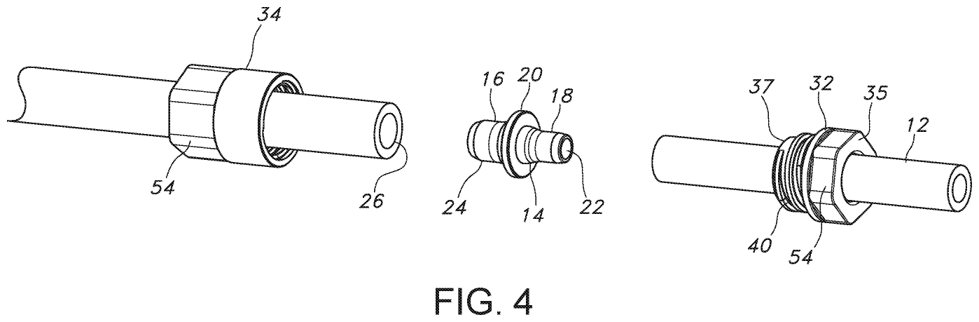

FIG. 5 is a perspective view of connector being placed between the tubing pieces.

FIG. 6 is a cross-sectional view of tubing pieces joined by the connector.

FIG. 7 is a cross-sectional view of tubing pieces joined by the connector and secured together by the coupling members.

### DETAILED DESCRIPTION

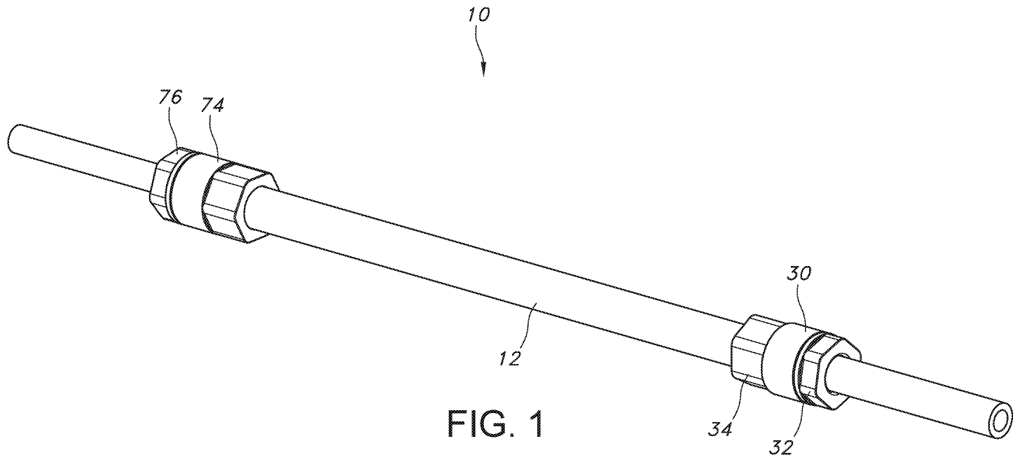

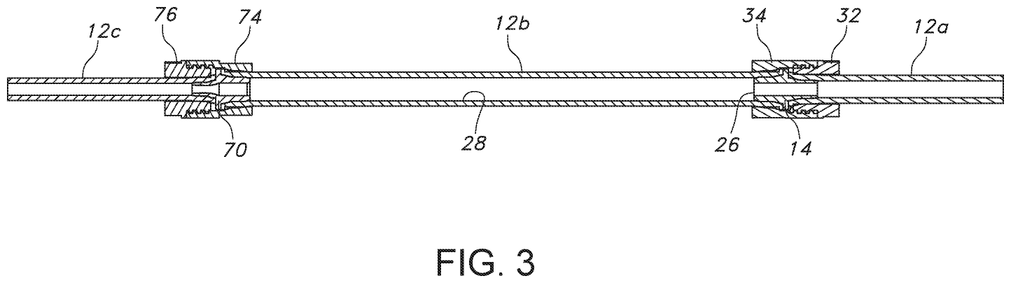

With reference to FIGS. 1-3, a connecting system 10 is shown. The system 10 joins together flexible tubing pieces, collectively identified as reference number 12, of either similar or dissimilar sizes in a manner which provides a secure connection that may be easily and repeatable removed and re-secured. The connecting system 10 may have a wide variety of application and is especially useful in laboratory and medical applications where secure connections of the flexible tubing are important to prevent leakage.

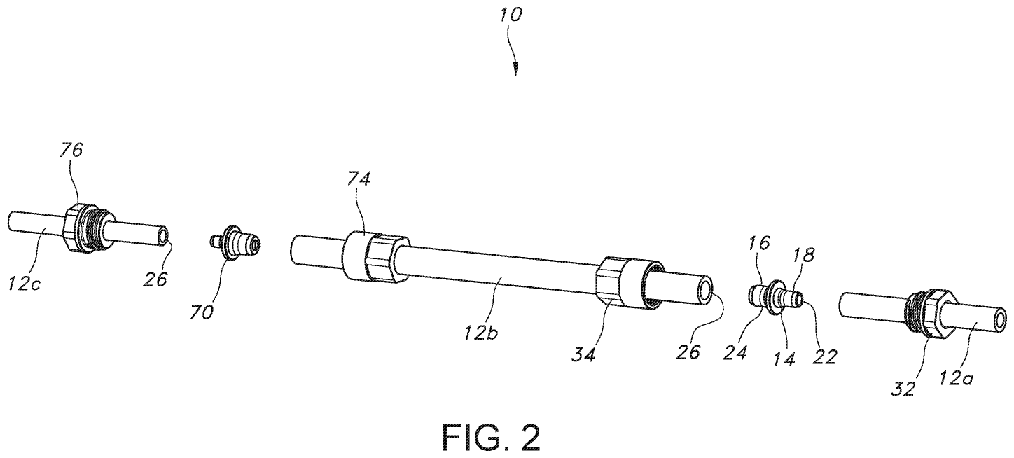

With reference to FIGS. 2, 3 and 5, the system 10 includes a first connector 14 including first and second cylindrical projections 16 and 18 extending in opposite directions from a central body 20. A lumen 22 extends though the first and second projections and body. Each of the first and second projections 16 and 18 includes a smooth surface, which has a decreasing diameter as it extends outwardly from the body 20. The change in outer diameter is achieved by way of a stepped taper that permits the tubing to slide there over. The first and second projections do not include barbs. The first and second projections 16 and 18 may have different outer diameters to permit tubes of different inside diameters to be joined. The first connector 14 may be formed of a plastic material such as a polycarbonate-ABS or other material known in the art.

The first and second projections 16 and 18 are inserted into the tubing openings 26 until the end of the tubing abuts the central body 20 which acts as a stopper. The outer surface has a stepped taper 24 and increases in diameter and facilitates the sliding of the tubing into the first and second projections. Therefore the first and second projections each include portions having differing outer diameter sizes. The projections may be sized to be slightly larger in outer diameter than the inner diameter of the tubing. The tubing may be formed of a flexible material, e.g., PVC, PU, nylon, silicone, or rubber, such that it expands and elastically engages the connector. In one embodiment, the first projection 16 may have an outer diameter greater than the outer diameter for the second projection 18. This permits tubing of different internal diameters to be connected together.

The system 10 further includes a first coupling 30 including a first coupling member 32 and a second coupling member 34. The first and second coupling members 32 and 34 may be joined and secured together. The first coupling member 32 includes a body 36 having an aperture 38 extending therethrough along its length. The aperture is defined by an annular sidewall 33 extending the length of the first coupling member 32. The first coupling member body 36 includes a joining element in the form of an externally threaded portion 40. The first coupling member has a first end 35 for receiving the tubing 12 and a second end 37 from which the tubing exits.

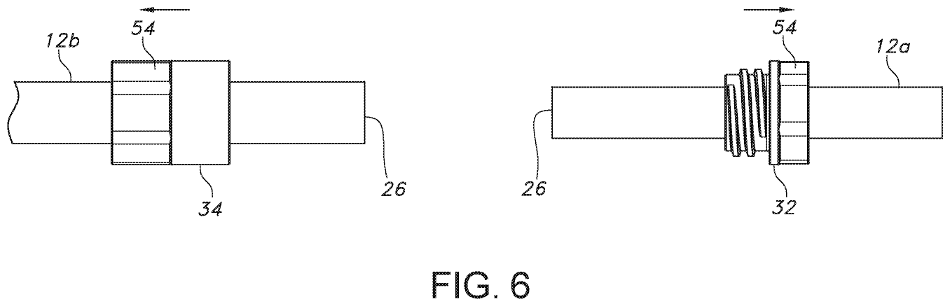

The second coupling member 34 includes a body 46 having an aperture 48 extending therethrough along its length. The aperture 48 is defined by a first annular sidewall 39 having a diameter sized to closely receive the tubing 12 therethrough and a second annular sidewall 41 having a diameter larger than the diameter of first annular sidewall. An annular step 43 is formed between the first and second annular sidewalls and forms an annular edge 45. The second coupling member body 46 includes a joining element in the form of an internally threaded portion 50 that threadedly receives the first coupling member external threads 40, thus joining the first and second coupling members together. While a threaded portions are shown and described herein for securing the first and second coupling members together, it is contemplated that other joining elements and devices could be used, for example, bayonet connections, snap connections, interlocking resilient members, clips, etc. As shown in FIG. 6, the first and second coupling members 32 and 34 are slidable along the tubing that extends though their respective apertures 38 and 48. The coupling member bodies 36 and 46 may each also include a gripping portion 54 that permits the respective members to 12 be gripped by hand or a tool to facilitate threading the first and second coupling members 32 and 34 together. In one embodiment, the gripping portion 54 could be a hex configuration. Alternatively, it is contemplated that gripping portion 54 could be a knurled surface or other surface to facilitate secure holding thereof.

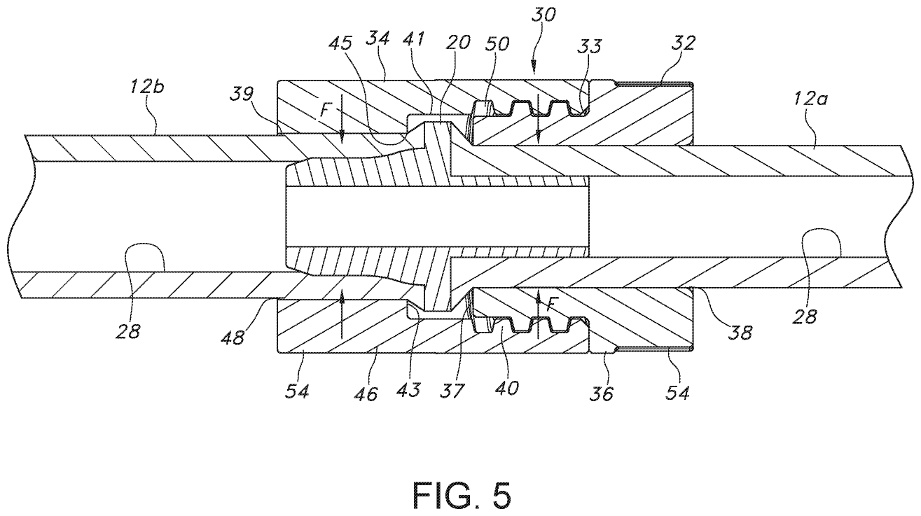

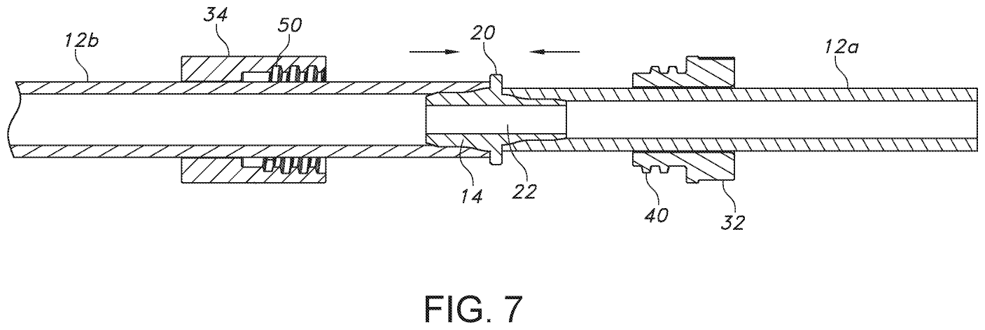

In order to secure two pieces of tubing 12a and 12b together, the first and second coupling members 32 and 34 are slid onto their respective piece of tubing as shown in FIG. 6. The tubing ends 26 are then urged onto the first and second connector projections 16 and 18 as shown in FIG. 7. This insertion causes the tubing ends 26 to expand, thereby increasing the outer diameter of the tubing ends. The increased diameter is greater than the diameter of the coupling aperture 38 and 48 through which the tubing extends. The first and second coupling members 32 and 34 may then be slid along the tubing toward each other until the outer thread 40 engages the internally threaded portion 50. The first and second coupling members 32 and 34 may then be rotated relative to each other to thread the inner and outer members together as shown in FIG. 5. As the first and second coupling members are drawn together, the annular sidewalls 33 and 39 defining the aperture of the first and second coupling members engage the tubing outer diameter. This exerts a hoop force F on the tubing that urges the tubing against the respective first and second projections. The joined pieces of tubing 12a and 12b are therefore securely retained on the first connector 14 and secured together in fluid communication with each other. In addition, the second coupling member edge 45 engages and elastically deforms, or pinches, the tubing 12b outer surface and urges the tubing 12b toward and against the central body 20. The first coupling second end 37 engages and elastically deforms the tubing 12a extending there through and urges the tubing 12a into engagement against the central body 20. Accordingly, the action of threading the first and second coupling members 32, 34 together both urges the tubing pieces 12 fully onto the connector 14 and exerts a hoop force F to sealingly secure the tubing onto the connector.

The tubing pieces 12 can be separated by unthreading the first coupling member 32 from the second coupling member 34 and sliding them apart. The pieces of tubing 12a and 12b may then be pulled apart from the first connector 14.

It is contemplated that the connectors and couplings can be formed in a variety of sizes to accommodate various sizes of tubing. As shown in FIGS. 1-3, a plurality of tube pieces 12a, 12b and 12c each having different sizes can all be joined together. Thus, the connector system 10 can accommodate a wide range of tubing sizes. For example, three pieces of tubing 12a, 12b, and 12c each of different sizes, e.g. different inner diameters may be joined together. The system 10 may include two connectors, each having the ability to join tubing pieces of different sizes. The first connector 14 joins tubing pieces 12a and 12b as described above. In a similar manner, the second connector 70 joins tube piece 12b to 12c. Two sets of couplings may also be provided. The first set includes first and second coupling members 32 and 34, which is used in combination with the first connector 14 for securing together tube pieces 12a to 12b. A second coupling 72 including third and fourth coupling member 74 and 76 are used with the second connector 70 that joins tubing 12a to 12c in a similar manner as described above.

It is further contemplated that a member of connectors and couplings sets could be provided in the system to permit tubing of more than three different sizes to be joined. In order to accommodate the various tubing sizes, connectors may be formed with different sized projections and couplings can be formed with different sizes apertures. The connectors and couplings may be formed as described above but with different dimensions to accommodate the particular tubing being joined together.

It will be appreciated that various of the above-disclosed features and functions, or alternatives thereof, may be desirably combined into many other different apparatus and systems. It will also be appreciated that various presently unforeseen or unanticipated alternatives, modifications, variations, or improvements therein may be subsequently made by those skilled in the art which are also intended to be encompassed by the disclosed embodiments and the following claims.

What is claimed is:

1. A system for joining tubing of different sizes comprising:
a first connector including a first projection and a second projection and the first connector including a lumen extending therethrough, the first projection having an outer diameter larger than an outer diameter of the second projection;
a first coupling including a first and a second coupling member, the first coupling member having a first body including a first aperture extending therethrough along a length thereof, the first body including threads, the second coupling member including a second body having a second aperture extending therethrough along a length thereof, the second body including threads complimentary with the threads of the first coupling member permitting the first and second member to be threadedly engaged, and the first aperture having a diameter larger than a diameter of the second aperture.

2. The system as defined in claim 1, wherein the inner and outer coupling members being slidable along tubing that extends though their respective apertures.

3. The system as defined in claim 1, wherein the first and second apertures are sized to closely fit the tubing extending therethrough.

4. The system as defined in claim 1, wherein the connector first and second projections each include portions having differing outer diameter sizes.

5. The system as defined in claim 3, wherein the first and second coupling members are adapted to clamp the tubing extending therethrough on the first and second projections when the first and second couplings are threadedly engaged.

6. The system as defined in claim 1, wherein the first and second coupling members include a gripping portion to facilitate holding.

7. The system as defined in claim 1, further including a second connector having a third and a fourth projection and a lumen extending therethrough,

8. The system as defined in claim 7, wherein the third projection has an outer diameter substantially similar to the second projection and the fourth projection has an outer diameter different from the outer diameter of the first and second projections.

9. The system as defined in claim 8, further including a second coupling having a third and a fourth coupling member, the third coupling member having an aperture therethrough sized substantially the same as the aperture in the second coupling member, and the fourth coupling member having an aperture.

10. The system as defined in claim 7, wherein the third and fourth projections each include a non-barbed surface.

11. The system as defined in claim 1, wherein the first coupling member includes an annular edge in the first aperture adapted to engage the tubing to urge the tubing to a stop formed on the first connector when the first and second coupling members are threadedly engaged.

12. The system as defined in claim 11, wherein the first aperture in formed by a first and second annular sidewall, and the first annular sidewall having a first diameter sized to closely fit the tubing extending therethrough and the second annular sidewall having a diameter larger than the first diameter and the annular edge is formed by a step between the first and second annular sidewalls.

13. A system for joining tubing of different sizes comprising:
a first connector including first and second projection and a lumen extending therethrough, the first projection having an outer diameter larger than an outer diameter of the second projection;
a second connector having a third and fourth projection and a lumen extending therethrough, the third projection having an outer diameter substantially similar to the outer diameter of the second projection and the fourth projection having an outer diameter different from the outer diameter of the first and second projections;
a first coupling including a first and second coupling member, the first coupling member including a first aperture extending therethrough along a length thereof, the first coupling member having a first joining element, the second coupling member including a second aperture extending therethrough along a length thereof, the second coupling member including a second joining element complimentary with the first joining element of the first coupling member permitting the first and second coupling members to be secured together, and the first aperture having a diameter larger than a diameter of the second aperture; and
a second coupling having a third and a fourth coupling member, the third coupling member having an aperture therethrough with a diameter substantially the same as the diameter of the aperture in the second coupling member, and the fourth coupling member having an aperture with a diameter different from the diameter of the apertures in the first, second, and third coupling members.

14. The system as defined in claim 13, wherein the first and second coupling members each have an annular edge adapted to engage tubing to urge the tubing onto the first connector first and second projections when the first and second coupling members are secured together.

15. The system as defined in claim 13, wherein the first and second projection each have a non-barbed outer surface.

16. The system as defined in claim 13, wherein the first joining element includes threads and the second joining element included threads complimentary with the threads of the first joining portion of the first coupling member for securing together the first and the second coupling members.

17. A method for joining tubing of different sizes comprising:
providing a first connector including first and second projection and a lumen extending therethrough, the first projection having an outer diameter larger than an outer diameter of the second projection;
providing a first coupling including a first and second coupling member, the first coupling member including a first aperture extending therethrough along a length thereof, the first coupling member having a threaded portion, the second coupling member including a second aperture extending therethrough along a length thereof, the second coupling member including a threaded portion complimentary with the threads of the first coupling member threaded portion permitting the first and second coupling members to be threadedly engaged, and the first aperture having a diameter larger than a diameter of the second aperture;
inserting a first tube first end into the first projection;
inserting a second tube first onto the second projection, wherein the second tube has an inner diameter size different that an inner diameter of the first tube;
inserting the first tube through the aperture in the first coupling member;
inserting the second tube through the aperture in the second coupling member;
advancing the first coupling and second coupling member and threading the first coupling and second coupling member together to secure the first tube and second tube to the first connector.

18. The method as defined in claim 17, including providing a second connector having a third and fourth projection and a lumen extending therethrough, the third projection having an outer diameter substantially similar to the outer diameter of the second projection and the fourth projection having an outer diameter different from the outer diameter of the first and second projections;
inserting a second end of the second tube onto the third projection and inserting a third tube onto the fourth projection, wherein the third tube has an inside diameter different from the inside diameter of the second tubing and inside diameter of the first tubing.

19. The method as defined in claim 18, including providing a second coupling having a third and a fourth coupling member, the third coupling member having an aperture therethrough with a diameter substantially the same as the diameter of the aperture in the second coupling member, and the fourth coupling member having an aperture with a diameter different from the diameter of the apertures in the first, second, and third coupling members;
inserting a first end of a third tube through the aperture in the fourth coupling member;
inserting the second tube second end through the aperture in the second coupling member; and
advancing the third coupling and fourth coupling member and threading the third coupling and fourth coupling member together to secure the second tube and third tube to the second connector and wherein the first, second and third tube are in fluid communication with each other.
